(19) United States
(12) Patent Application Publication
(10) Pub. No.: US 2021/0387027 A1
(43) Pub. Date: Dec. 16, 2021

(54) Ventilation and Audio Mask Assembly

(71) Applicant: Alan J Mittelman, Calabasas, CA (US)

(72) Inventor: Alan J Mittelman, Calabasas, CA (US)

(21) Appl. No.: 17/243,245

(22) Filed: Apr. 28, 2021

Related U.S. Application Data
(60) Provisional application No. 63/017,142, filed on Apr. 29, 2020.
(60) Provisional application No. 63/038,585, filed on Jun. 12, 2020.
(60) Provisional application No. 63/047,352, filed on Jul. 2, 2020.

(51) Int. Cl. A62B 18/02; A41D 13/11; A62B 23/02; A62B 18/08

(52) U.S. Cl. CPC A62B 18/025; A41D 13/1107; A62B 18/08; A62B 23/02; A41D 13/1161

(57) ABSTRACT

A face mask assembly comprises a face mask for placing on the face of a user so as to define a space between the face mask and the face of the user. A ventilation component is associated with the face mask. The ventilation component has an intake port, a discharge port, a pump for conveying air through a conduit from the intake port to the discharge port, an air filter in the conduit, and a power source for the pump. The discharge port of the ventilation component connects to the face mask so as to deliver air to the space. The face mask assembly further comprises a sound enhancement assembly.

### CROSS REFERENCE TO RELATED APPLICATIONS

This application claims the benefit Of U.S. Provisional Patent Application No. 63/017,142 filed Apr. 29, 2020, Provisional Patent Application No. 63/038,585 filed Jun. 12, 2020, and Provisional Patent Application No. 63/047,352 filed Jul. 2, 2020, all of which are incorporated herein by reference in their entireties.

### FIELD AND BACKGROUND OF THE INVENTION

This invention relates to a ventilator and audio mask assembly. More particularly, the invention is for a mask, used in a health protective context, which is placed over the face, and particularly the nose and mouth, in order to protect the user from the intake of germs, viruses, pollutants, smoke or other impurities or undesirable particulate matter which may be in the ambient atmosphere.

It has become more common, and even prevalent, for many people, particularly including those involved in providing healthcare, to use face masks when venturing out into public spaces or other areas where a plurality of people may gather. As an example, just about every country on earth today has been impacted by the coronavirus, and the covid 19 infection, a potentially deadly virus which has caused a worldwide pandemic. People have therefore become much more attuned to the wearing of face masks, which offer some protection against transmission of the virus, through airborne transfer, when breathing the air in the area of a person who is infected, and who may have coughed, sneezed or otherwise discharged droplets or matter containing the coronavirus. The use of a face mask is therefore a critical action which a person can take in order to protect herself from infection and to the potential ravages of the coronavirus or, indeed, any other virus or germ which may be present in the air. A mask may also help to stop the wearer from touching her nose, mouth or eyes, through which infection may occur.

It is common, however, for some people to encounter problems while wearing a face mask. Such problems may often be exacerbated when the user is also wearing goggles or eyeglasses in conjunction with the mask, a not unusual situation. Since the face mask is often reasonably well, if not completely, sealed against the face of the user, breath from exhalation may be discharged into and around the edges of the face mask, including that area which may be near or adjacent to the goggles or eyeglasses which the user is wearing. In such instances, it may often happen that the exhalation of breath causes the inside surface of the face mask or eyeglasses to become fogged up, which has the annoying consequence of the user's vision being impaired, at least temporarily, until such time as the condensation of breath on the goggles or eyeglasses dissipates.

The presence of a face mask on the user also has the effect of damping or distorting voice sounds, making it often more difficult to understand the speech of the user, causing inconvenience and potential miscommunication.

However, the exhalation of breath is, of course, an ongoing occurrence and even if the fogged-up interior of the goggles or eyeglasses has cleared before the subsequent exhalation, it will fog up again when the next breath is exhaled.

Worse still, if the condensation has not cleared before the next breath exhalation, the inside of the goggles or eyeglasses will become even more fogged up, leading to increasingly cloudy and blurred vision of the user. This may become a serious impediment to those relying on good vision for carrying out their activities.

### SUMMARY OF THE INVENTION

In accordance with one aspect of the invention, there is therefore provided a ventilated mask assembly which is designed to address the problems mentioned above, and to reduce or eliminate the possibility of condensation and fogging of the eyeglass lens, as well improve speech communication. In one embodiment, the ventilated and audio mask assembly of the invention comprises a face mask for wearing over the face of the user, and a pump or aerator or air intake which may be preferably powered by a portable power source. The face mask and the pump or aerator are connected, preferably in a releasable manner, to each other by a tube or conduit which conveys air from the pump to the inside of the face mask. The discharge of air into the inside of the face mask, or the space between the inner surface of the mask and the face of the user, helps to ventilate this space, keeping it cool and reducing or eliminating condensate from settling on certain surfaces. Such surface will typically be a plastic mask shield which may be worn by the user, or goggles or eyeglasses worn by the user.

Preferably the power source is a portable charger, and may of the type of portable charger commonly used to charge smart phones or other portable electronic devices when the user is away from a more traditional charger, such as a computer or electric outlet in a structure. The portable charger is electrically connected to the pump or aerator, such as by a cable in one version, such as a micro USB cable, a Type C cable, or other type of cable or connector which perform the necessary function.

In another embodiment, the assembly may comprise a module attached to or incorporated into a mask or goggles, the module including an air intake and filter, a power source, and a mechanism for introducing air in to the space between the mask and the user's face.

Preferably, the portable charger can be disconnected from the pump or aerator when the power therefrom has been depleted, and replaced with another fully charged portable charger. The ventilated mask assembly of the invention is thus available for use for as long as needed, and continues to function with replaceable power sources when needed. This is useful for, as an example, certain health care workers who may need to wear the face mask for a long period or shift. Carrying a spare power source which can replace the power depleted one can be easily achieved by the user. It is also useful when the wearer is busy with outdoor or field work, when no power charger is readily available.

In another version or embodiment, the power source is fixed to the pump or motor and may be recharged by connecting it to a computer or fixed power source when needed. A power source of the type which may be used in this invention will have a long life since the pump or aerator is generally quite small and will require only a small amount of power to keep it running. Thus, a portable charger will preferably have plenty of power to keep the aerator or pump operating for an extended period of time.

Other ventilation mask assemblies of the invention may include both the ability to remove and replace a power source, as well as the ability to charge it when connected to the pump or motor when a power charging source is available and the pump or aerator is needed at that time.

According to one aspect of the invention, there is provided a ventilated mask assembly comprising a housing, and air conduit within the housing, a pump for conveying air through the air conduit, a replaceable power source for providing power to the pump, and a tube connector or other type of connector for moving air from the air conduit to a face mask. Preferably, the ventilated mask assembly of the invention includes a filter for filtering and purifying air taken in by the assembly. Further, the ventilated mask assembly may include the face mask itself, which is configured and adapted for use on a wearer, and for receiving a pumped and purified air supply from the housing.

According to another aspect of the invention, there is provided and ventilated and audio mask assembly comprising a face mask or goggles, a microphone for receiving sounds from the wearer, and a connector or transmitter for conveying the sounds from the wearer to a speaker either on the face mask or goggles or remotely therefrom.

### BRIEF DESCRIPTION OF THE DRAWINGS

FIG. 1 is a schematic representation of a ventilated mask assembly in accordance with one aspect of the present invention;

FIG. 2 is a schematic view of the inside of a mask of the invention including one embodiment of a microphone;

FIG. 3 is a schematic view of the outside of a mask of the invention including one embodiment of a speaker;

FIG. 4A is a perspective exploded view of a face mask and audio enhancement system in accordance with one aspect of the invention;

FIG. 4B is a perspective rearview of the face mask in FIG. 4A, including the speaker covers;

FIG. 4C is a perspective rearview of a face mask as shown in FIG. 4A, without the speaker covers;

FIG. 4D is a front perspective view of a face mask and audio enhancement system as shown in FIG. 4A of the drawings;

FIG. 5 is a front perspective view of a further embodiment of the invention showing a face mask or goggles with a ventilation module attached thereto;

FIG. 6 is a side perspective view of the face goggles illustrated in FIG. 5, showing the ventilation module;

FIG. 7 is a rear perspective view of the face goggles illustrated in FIG. 5, showing the ventilation module;

FIG. 8 is a top perspective view showing the face goggles illustrated in FIG. 5, and details of the ventilation module mounted thereon;

FIG. 9 is a top view of the face goggles illustrated in FIG. 5, showing the ventilation module when disconnected or disassembled from the goggles; and

FIG. 10 shows a circuit diagram indicating one embodiment of the electronic components and their relationship of a voice enhancement and amplifier system in accordance with the invention.

### DETAILED DESCRIPTION OF THE INVENTION

Reference is now made to the accompanying drawings, which show several embodiments of a ventilated and audio system mask assembly in accordance with the present invention. These accompanying figures show schematically and diagrammatically the various components which may comprise the ventilated mask and audio assembly of the invention, and these may be configured in different formats, designs and structures without derogating from the purpose and objective of the invention.

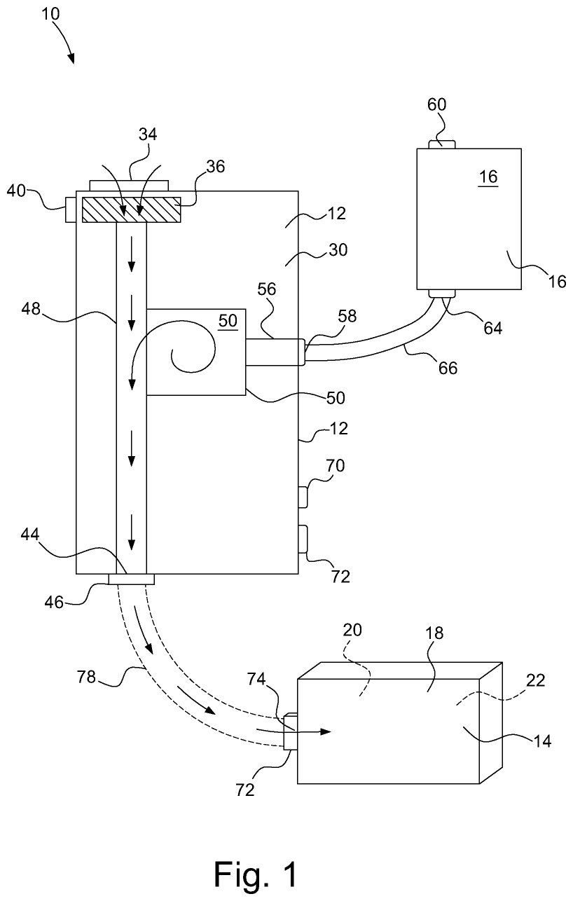

FIG. 1 of the drawings shows a ventilated mask assembly 10. The ventilated mask assembly 10 comprises a housing 12, a face mask 14, and a power source 16. The face mask 14 is represented schematically, and will typically have attachments, such as straps, ties or elastic bands, for securing the mask 14 over the mouth and nose of the user by looping the straps or elastic bands around the head or neck, or around and behind the ears of the user. The face mask 14 comprises an outer surface 18 and an inner surface 20. The shape of the face mask 14 is such that a ventilation space 22 is formed inside the mask 14, so that at least a part of the inner surface 20 will be spaced from the mouth, nose and face of the user. Depending upon the mask 14, the ventilation space 22 may be relatively, but not completely, sealed so that only a small amount of air may be allowed into and out of such ventilation space 22. Alternatively, the mask 14 maybe a little looser or more flexible with more such areas along the edge thereof through which air may pass between the ventilation space 22 and the outside. The ventilation space 22 may vary in size during use, and need not be of any significant size or volume.

The housing 12 is shown in the schematic FIG. 1 as being somewhat rectangular, but the invention is not limited to such a structural configuration. The housing 12 maybe any convenient or suitable shape for carrying out its function, and may be comprised of any hard and generally unbendable, inflexible material, a flexible material, or a combination of both.

The housing 12 comprises an outer shell 30 defining an interior 32, which is generally sealed with the exceptions set forth below. The outer shell 30 has an opening or intake port 34 at a location along its surface, through which ambient air is taken into the housing 12. Adjacent the intake port 34, there is provided a filter 36 which may be positioned in a filter compartment or capture area, through which all air entering the housing 12 must pass. The filter 36 may be selected according to, for example, the context in which the ventilated mask assembly 10 may be used. As such, certain filters may be more preferable when the ventilator mask assembly 10 is used in particulate, smoky or polluted conditions, as compared with a different filter which may be used in a medical or health setting where the mask is intended to protect the user against airborne viruses or germs in the ambient atmosphere. It will be noted that the same ventilator mask assembly 10 can be used in different settings, and the filter 36 selected and exchanged depending upon the needs of the user and the external conditions which may require that the surrounding air of be filtered.

A filter door 40 may be provided to allow easy access to the filter 36 and filter compartment, so that it can be easily replaced or cleaned when dirty, or when a different type of filter 36 may be needed based on outside conditions.

The housing 12 further comprises a discharge outlet 44 at a location along its outer surface. The discharge outlet 44 has adjacent thereto, and on the outside of the housing 12, a tube connector 46, for connecting to a tube as will be described below. Within the housing 12, and extending between the intake port 34 and the discharge outlet 44, there is provided an air conduit 48. The air conduit 48 is sealed between these two ends, and its purpose is to convey a stream of air from the intake port 34, through the filter 36, and out of the discharge outlet 44.

A pump or motor 50 is provided within the interior 32 of the housing 12. The pump 50 is associated with the air conduit 48 so that activation of the pump 50 causes air to be sucked in or drawn in through the intake port 34, conveyed along the air conduit 48, and discharge through the discharge outlet 44.

In practice, the pump 50 may be somewhat small in size and capacity, since, in practice, only a small stream of air need be generated for the purpose of satisfying the operation and function of the insulated mask assembly 10. However, the invention is not limited based on the size of the pump or motor 50 or flow rate of the air generated by the system, and these factors will be taken into account when constructing the ventilated mask assembly 10 of the invention for the intended purpose.

The pump 50 is fixed within the housing 12 by means of a bracket or fastener 56. The bracket or fastener 56 may also include or support the necessary electrical connector components, so that the pump 50 can be connected to a source of power 16, as will be described. An electrical connector port 58, which may be in the form of a USB port but is not limited thereto, is located along the outer wall of the housing 12, so as to connect to the pump 50.

The ventilated mask assembly 10 includes a power source 16. The power source 16 will typically comprise a portable battery or charger, and preferably a portable battery which can be charged and recharged multiple times. Such batteries are reasonably well known currently, and may typically be used to charge mobile electronic devices including smart phones, tablets, to name two examples. Such a power source 16 will, in use, include a charging port 60, by means of which the power source 16 may be releasably connected for charging to a source of power, such as a computer or any electrical outlet. Such a power source 16 may generally take about an hour, or a few hours, to fully charge, and may thereafter be connected to a mobile electronic device to provide it with enough power so that the mobile electronic device can continue to function for hours or even days, depending upon the nature of the mobile electronic device, and the amount of power which it needs to operate.

The power source 16 further comprises a connector port 64. A connector cable 66 extends between the electrical connector port 58 on the housing 12 and the connector port 64 on the power source 16. When so connected as needed for charging, the power source 16 provides the necessary power to selectively activate and deactivate the pump 50, on an as needed basis. In one embodiment, the power source 16 may be located within the housing 12, to provide a more compact unit.

The pump 50 itself may be a variable pump so that the user can select the amount of air that the housing 12 will deliver to the face mask 14. The housing 12 will include an on/off switch 70 to activate the pump 50. The housing 12 may further comprise a switch or button 72 by means of which the airflow and speed of the pump may be adjusted. When the user may become more physically active, and there is an accompanying increase in cardiovascular demand, the air flow rate and speed of the pump can be adjusted so as to deliver more air into the ventilation space 22 of the face mask 14. The air which flows into the ventilation space 22 will not only provide additional oxygen to the user, but also facilitate a reduction or even elimination of any fogging up which may occur on goggles or eyeglasses which the user is wearing, thereby reducing the inconvenience and annoyance of impaired vision as a consequence of condensation from warm breath on the inside surface of such goggles or eyeglasses.

Additionally, the ventilated mask assembly 10 may deliver cooler air to the ventilation space 22, also adding to the comfort of the wearer. There are certainly conditions in which the temperature of air within the ventilation space 22 can increase, with exhaled breath usually being warmer than the ambient surrounding air, and thereby become unpleasant for the user, and the delivery of cooler air to counterbalance this adds to the potential advantages of the ventilation mask assembly 10 in accordance with the present invention.

The various components of the ventilation mask as described above may, in one embodiment, be incorporated into a single module which may attach to the face mask itself. In such an embodiment, the tube 78 may be dispensed with, in whole or in part, since the air outlet of the pump mechanism may be directly adjacent an input aperture or valve on the mask. All of the essential components, including filter, motor or pump, and power source may be integrated into a single module. An example of such an embodiment is described and illustrated below.

In operation, the housing 12 of the ventilated mask assembly 10 may hook onto, for example, a belt or waistband of the user. It is preferably small and light and compact, and designed to be so in order that it not impede or obstruct the movements of the wearer. The housing 12 of the assembly 10 may be no larger than, for example, a wallet or similar sized personal items worn by the user.

The housing 12 is connected to the power source 16 by connecting the connector cable 66 between the electrical connector port 58 and the connector port 64 of the power source 16. When such a connection has been effected, a power supply has been put in place and the pump 50 may then be switched on and off, as needed or desired. It should be noted that the power source 16 is also very compact, and may be held or located away from and separately from the housing 12, or it may be clamped or otherwise connected thereto so they are conveniently maintained together as a single unit. However, this would not affect the ability of the user to remove the power source 16, when it has been depleted, and replace it with another. The depleted power source 16 can then be charged later so that two or more power sources can be appropriately rotated in order to ensure a constant source of power over a prolonged period, and operation of the ventilated mask assembly 10.

A long tube 78 can be releasably installed between the tube connector 46 at the discharge outlet 44 and the tube connector 72 on the mask 14. The tube connector 72 is associated with an aperture 74 in the mask 14. The tube 78 may be conveniently placed or fixed relative to the user so that it will not extend away from the body of the user, and hence become a nuisance or even a danger. Appropriate clamps, ties or connectors may be used, or hook and loop style connectors, such as Velcro®, may be used as securing mechanisms, and thereafter discarded, as necessary. The tube 78 is preferably connected to the discharge outlet 44 at one end, and the tube connector 72 at the other end, in a generally sealed manner so as to prevent any escape of air along the way or, by the opposite token, from drawing in air from the outside which has not previously been filtered through the filter 36.

With all of the components of the ventilator mask assembly 10 in place, the mask 14 may be placed around the face of the user, by straps, ties, elastic bands or other conventional mechanisms for securing the mask 14 to the face. At this time, the switch 70 can be activated, so that the pump 50 commences operation, drawing ambient air into the intake port 34, discharging it through the discharge outlet 44, and conducting it through the tube 78 to exit through the aperture 74 and into the ventilation space 22 formed between the face mask 14 and the face of the user. The user may adjust the flow speed and volume by appropriately manipulating the adjustment button 72, so that a comfortable working condition is achieved by providing sufficient filtered air into the mask 14 to reduce or prevent condensation, provide cool air, and increase the comfort of the user.

The invention may incorporate or comprise several additional features or structures. In one form of the invention, an existing mask may be retrofitted in order to make it compatible with a pump assembly of the invention. In this embodiment, a mask may be treated or changed so that it is punctured, or a small hole or aperture is made in the mask, preferably at a side location or in an off-center position, although any convenient position would work within the scope of the invention. The aperture may be rounded with clean edges. A connector is mounted on the mask at the location of the aperture, so as to cover, seal and surround the aperture. Such a connector may take several different forms. For example, it may be a two part snap-on connector wherein one part of the snap connector is on one side of the mask or mask fabric or material and the other part of the snap-on connector is on the opposite side of the fabric or material. The two parts of the connector snap together, with the fabric or material firmly sandwiched between them, and the aperture in the fabric or material of the mask is covered by the snap connector. Each part of the snap connector has a corresponding snap aperture defining a conduit or passageway through the snap connector. Furthermore, the aperture in the mask registers with the snap connector apertures, so that access through these apertures is provided, for the inflow of air, as described below.

As noted, the snap connector may have two parts, and one of these will be on the outside of the mask while the other will be on the inside thereof, with the mask fabric secured between the two. In one embodiment, a tube or hose attachment portion is configured as a part of the snap connector part which is on the outside of the mask fabric. A tube or hose may be releasably fastened to the tube attachment portion, and the tube or hose is connected or associated at its other end to the pump device, so that air, fresh or purified, may be conveyed from the pump device through the tube to the inside of the mask to provide the wearer with a steady stream of fresh and filtered air. In this manner, an aftermarket addition may be made to face masks without the air stream input, enhancing the safety and comfort of such masks.

The invention may also include mechanisms for voice enhancement and control. The audio enhancement and control may be in addition to the ventilation component of the mask assembly, or the mask assembly may have either a ventilation component or an audio enhancement component separately. In other words, the mask assembly may include a ventilation component, and audio enhancement component, or both of these components.

It will be apparent to most that wearing a mask over the mouth may impact upon the clarity and the quality of speech actually heard by the listener, and the presence of the mask will often make the sounds muffled or muted, and degrade the voice enunciation to make them harder to understand. The invention therefore comprises, in one aspect, a voice system which may be integrated into the mask. The voice system may comprise a microphone mounted on or within the mask, and the microphone may be powered by an inclusive battery; in another embodiment the microphone may be connected by electric wires to a power source, either one dedicated for the purposes of the microphone, or one that powers other components of the invention, such as the pump.

The microphone is, in one embodiment, preferably attached to the mask or face cover in such a way that its attachment creates no spaces or openings through which air exhaled by the wearer can pass. This may add to the safety characteristics of the mask or face cover. Additionally, or alternately, the mounting of the microphone may include filters so that any air exhaled by the wearer or expelled from the inside of the mask is filtered and particulate or other matter removed, again providing an additional level of safety.

The microphone is associated with a speaker of some sort, and the relationship and form of connection between the microphone and the speaker may take many different forms. In one embodiment, a speaker may be placed on the mask and electrically or wirelessly connected to the microphone. In another embodiment, the speaker may be on the person of the wearer, situated off the mask, and connected to the microphone, once more either electrically using a cable or wirelessly such as using a Bluetooth connection. In yet another embodiment, the speaker may be on or in a smart phone or other type of electronic device such as a tablet, and connected, additionally or instead of one of the other methods above, by a Bluetooth connection so that the voice of the face mask wearer is received on the smart phone or other such device. In this regard, it is also within the scope of the invention that the mask itself, or an accessory thereto, comprise a speaker which may be positioned near the ear(s) of the wearer so that a conversation or exchange may be had with a remote person.

Where Bluetooth or wireless forms of connection may be used, the devices would include transmission and receiving components for achieving the purpose of transmitting the voice of the wearer.

As noted from the above, there may be a number of different mechanisms for enhancing voice quality and transmission using a mask assembly of the invention. While only certain examples have been provided herein, the scope of the invention is not to be limited by the type of voice enhancement and/or transmission system which is used. The invention is intended to comprise a face mask which has mechanisms associated therewith for improving and enhancing the transmission of sound by allowing the voice of the wearer to be heard in ways other than directly from the mouth, especially that such sounds are often muted or muffled as a result of the presence of the face cover on the wearer.

The voice enhancement system when incorporated into the invention may take many different forms. Generally, the voice enhancement system may be one which is active, or one which is passive. A passive system may be one in which the voice is conveyed from inside the mask to a location outside or elsewhere on the mask by means of a mechanical structure, while an active system may require components of an electronic nature for processing the voice, as briefly described below.

Many such mechanical structures may be used and fall in the scope of the invention. The passive systems may comprise, as examples, tubes or conduits to pipe the voice from the inside to the outside of the mask. In one embodiment, appropriate acoustic structures may be present at one or both ends of the conduit to either receive the user's voice sounds, or broadcast or play the user's voice sounds, as applicable. In another embodiment, the passive system may be a diaphragm or other type of material built into the mask which allows the voice to be transmitted from the mouth of the user in the mask and through the fabric or substance of the mask, and wherein the diaphragm or other type of material has better sound transmitting characteristics than the fabric or substance from which the mask is manufactured. For example, a thinner material may be utilized, or a material which has less of a deadening or muffling effect may be incorporated. Such material would preferably be strategically placed within the mask, so that it is at or near the mouth when the mask is correctly positioned on the face of the wearer. The size and configuration of the material can also be adjusted in accordance with specific situations and requirements.

Active systems require that the voice of the user be electronically processed, such as by means of a microphone. The microphone may be connected by wire or Bluetooth or other form of wireless connection to a speaker. The speaker may be mounted on the user's mask, on another user's mask, or on a platform or substrate fastened or connected conveniently on the body of the user. In another embodiment, the speaker may be a device such as a smart phone, tablet or computer or other electronic device such as a digital assistant (Alexa or Google, for example). The voice sounds may be transmitted to a local area, or they may be transmitted over the internet to any selected remote location or device.

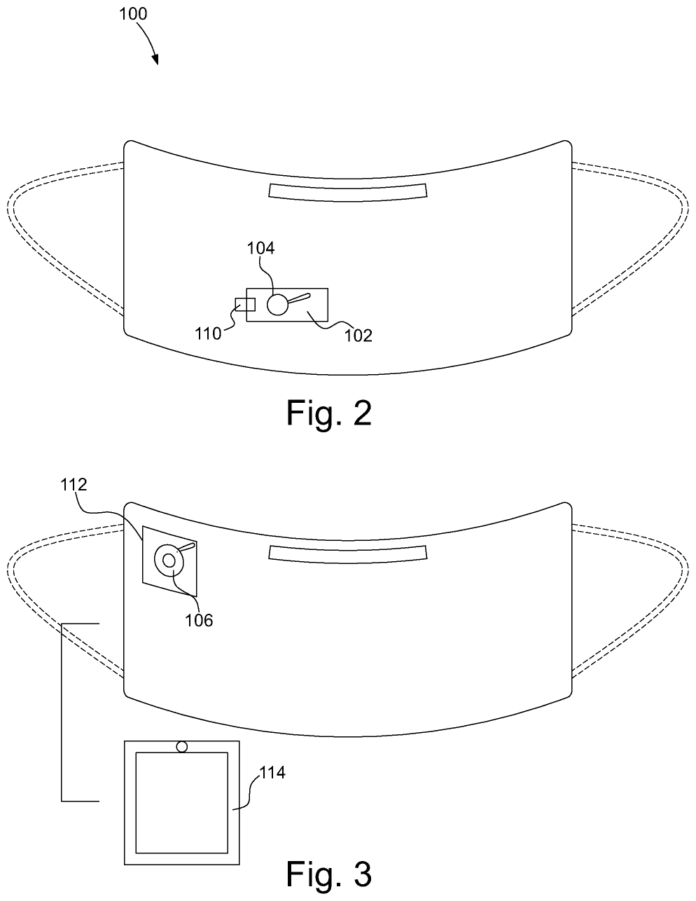

In FIGS. 2 and 3 of the drawings, one schematic embodiment of the mask of the invention is illustrated. These drawings are intended to be exemplary, and certainly not definitive of the possible ambit of the invention. FIG. 2 shows the inside view of a mask 100 with a pocket or compartment 102 for accommodating a microphone 104, which may have an associated transmitter, while FIG. 3 shows the outside view of a mask 100. The pocket or compartment 102 is preferably at or near the mouth of the user, but may be elsewhere on the mask 100. The microphone 104 may be removable so that it can be placed in another mask or removed for cleaning and then replaced. The microphone 104 may have the ability to transmit wirelessly to a speaker 106 or it may be wired to a speaker 106 on or near the mask 100, or it may have both of these functions.

The microphone 104 may be rechargeable with its own power source 110, or it may be connectable to a power source, such as the one powering the pump, as described above, if the mask incorporates this structure. Recharging of the microphone 104 may occur when the device is located in the mask 100 or face covering, or when it is removed. Preferably, there would be sufficient charge to keep the microphone 104 operational for extended periods of time.

FIG. 3 of the drawings shows the outside of a mask 100 with a pocket or compartment 112 in which is located a speaker 106 which may have a receiver associated therewith for receiving signals from the transmitter described above, or even from another transmitter. This figure also shows a smart device 114, such as a phone or tablet, which receives signals from the microphone 104 so that it can play the voice and sounds transmitted. The speaker 106 may be located on the mask 100 as shown in this figure, or elsewhere on the mask 100, or on the body of the wearer, or indeed remote from the body of the wearer. All of these options and others fall within the scope of the invention.

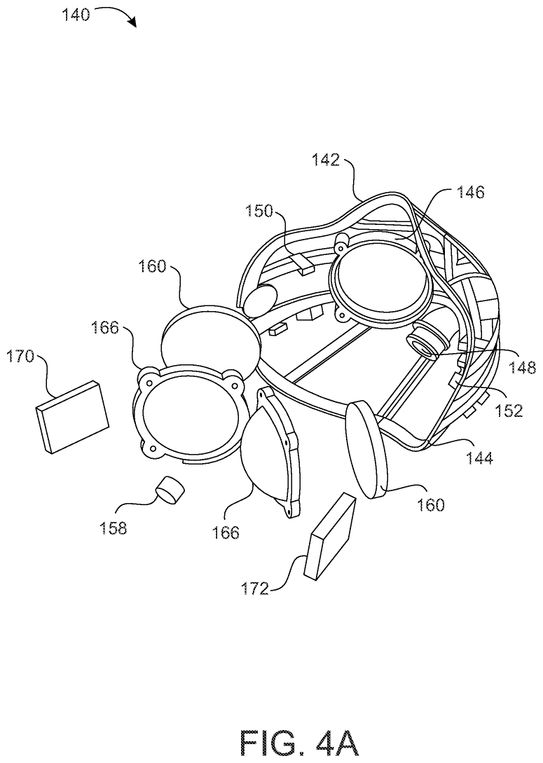

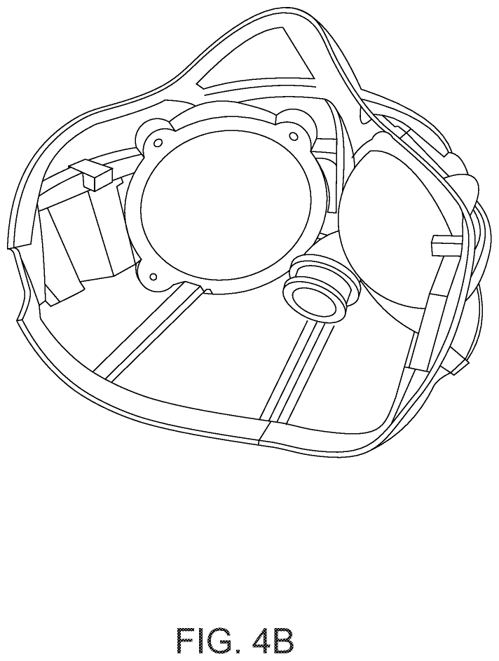

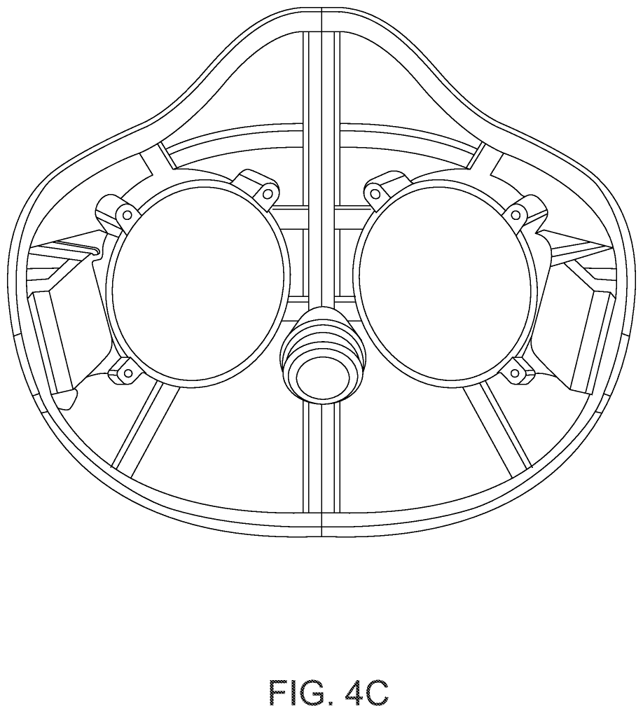

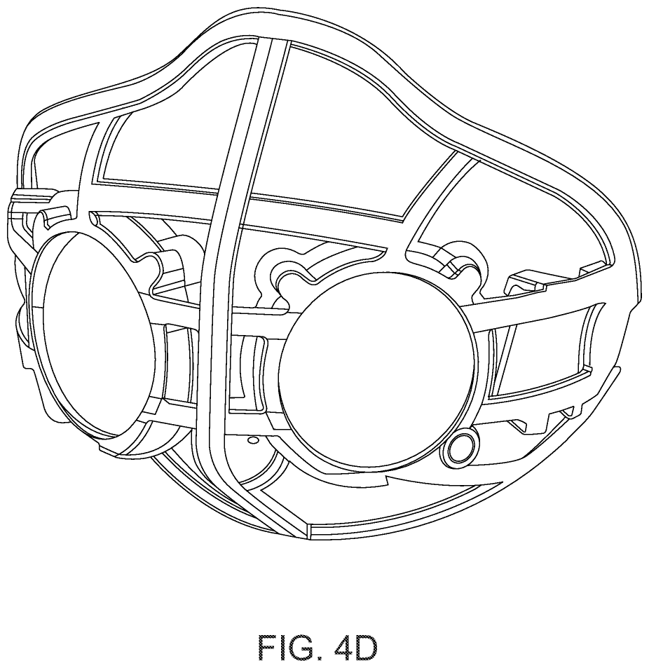

Reference is now made to FIGS. 4A to 4D of the drawings which show one embodiment of a mask including a sound enhancement system in accordance with the present invention. FIG. 4A illustrates an exploded rear perspective view of a mask in accordance with this aspect, while FIGS. 4B, 4C and 4D show, respectively, a rear perspective view including the speaker cover, a rear perspective view without the speaker cover, and a front perspective view of the device in accordance with the invention. The mask 140 in accordance with this embodiment includes a frame 142, the frame 142 having frame members 144 configured in the shape of a face mask, and which may be covered by appropriate fabric, plastic or other material to prevent passage of air from the outside of the mask frame to the inside thereof. The frame 140 includes a pair of circular brackets 146 and a microphone base 148. Further, the frame 140 includes clamps 150 for receiving a PCB, and a set of clamps 152 for receiving a battery 172.

The microphone 158 is mounted within the microphone base 148, and secured therein. This securement may be of a permanent nature, or one in which the microphone 158 can be easily removed and replaced for maintenance, or a more appropriate device in a given application. The pair of speakers 160 fit within the respective circular brackets 146, and are sealed between the circular brackets 146 and the speaker covers 166 so that they remain in place in a sealing configuration. In this way, each of the circular brackets 146 receives a speaker 160, securing in the designated space by screws or similar structures if necessary, in a sealing fashion so that air cannot pass through or around the speakers 160. Speaker covers 166 also secured to the circular brackets 146, holding the speakers 160 firmly in place.

A PCB 170 is fastened to the clamps 150, while a battery 172 is secured to the clamps 150 to hold the battery 172, preferably in a releasable manner so that the battery 172 can be removed and charged on an ongoing basis. Appropriate electrical and other connections are made between the PCB 170, as well as the battery 172, the speakers 160 and the microphone 158. As mentioned, the frame 142 is covered so as to define an inner area which is separated from the outer ambient surroundings.

The mask 140 as described and illustrated here in may also include an inlet for ventilation, so that the inside of the mask will receive a supply of air. The ventilation may be provided by an attached module mounted on the mask 140, or through a tube or other conduit attached to a motor or pump whereby air may be introduced into the inner area, in one of the manners described with reference to embodiments of the mask which have such ventilation.

Note that the voice enhancement systems in accordance with the invention, and as described herein, may further include a voice amplifier for amplifying the voice of the wearer, and potentially improving the corresponding sounds emanating from the speaker.

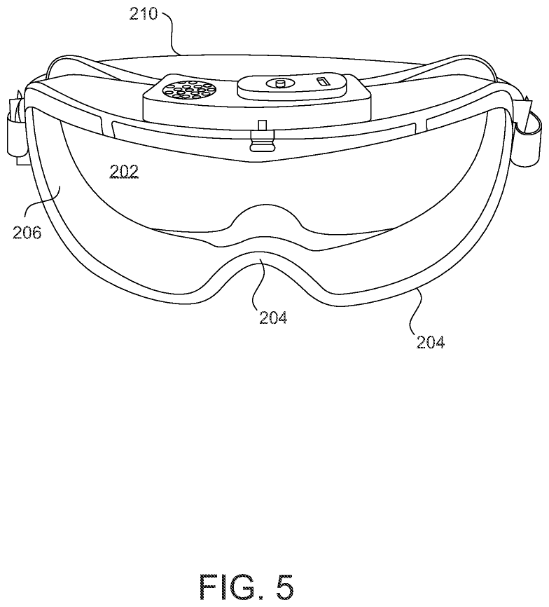

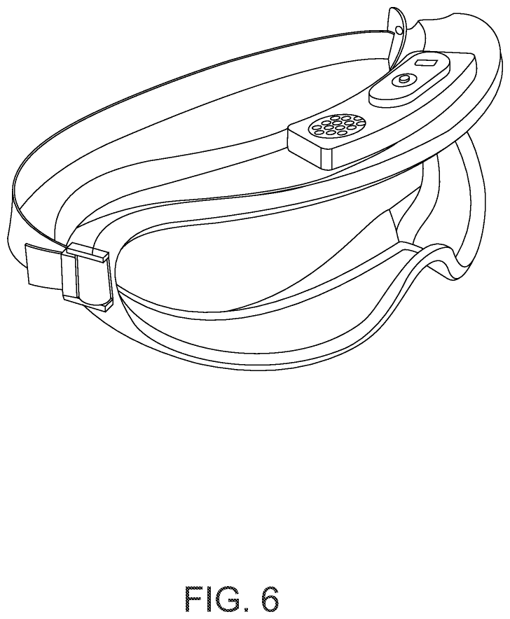

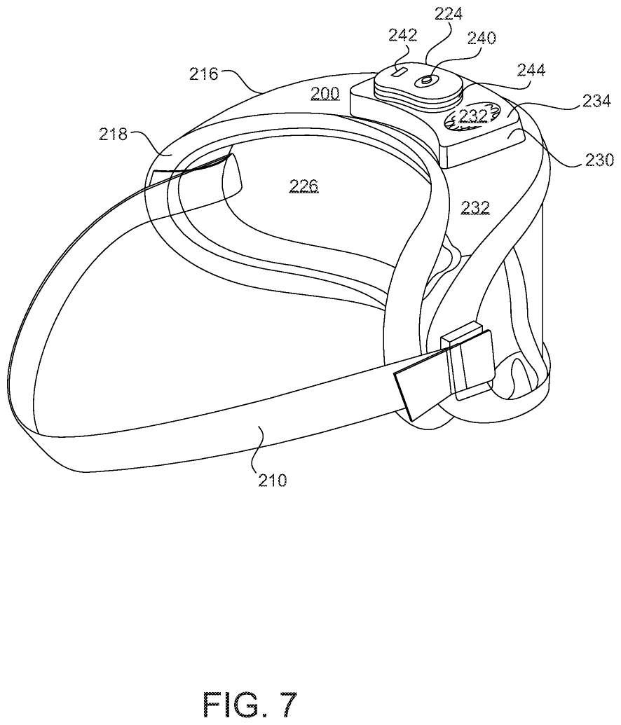

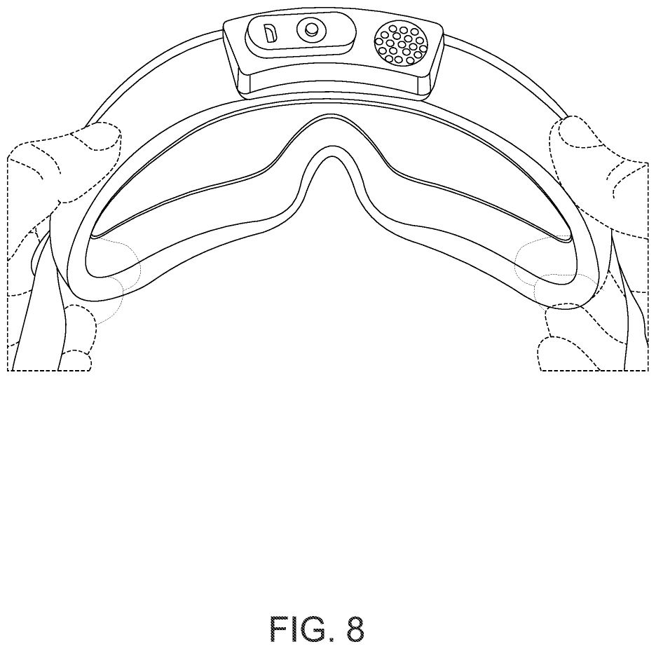

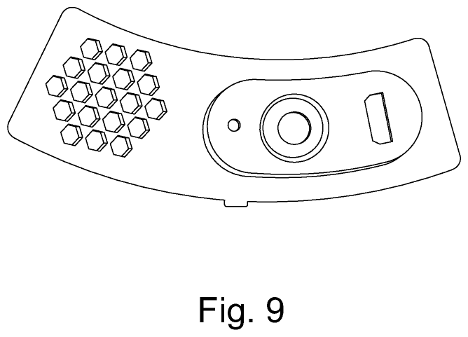

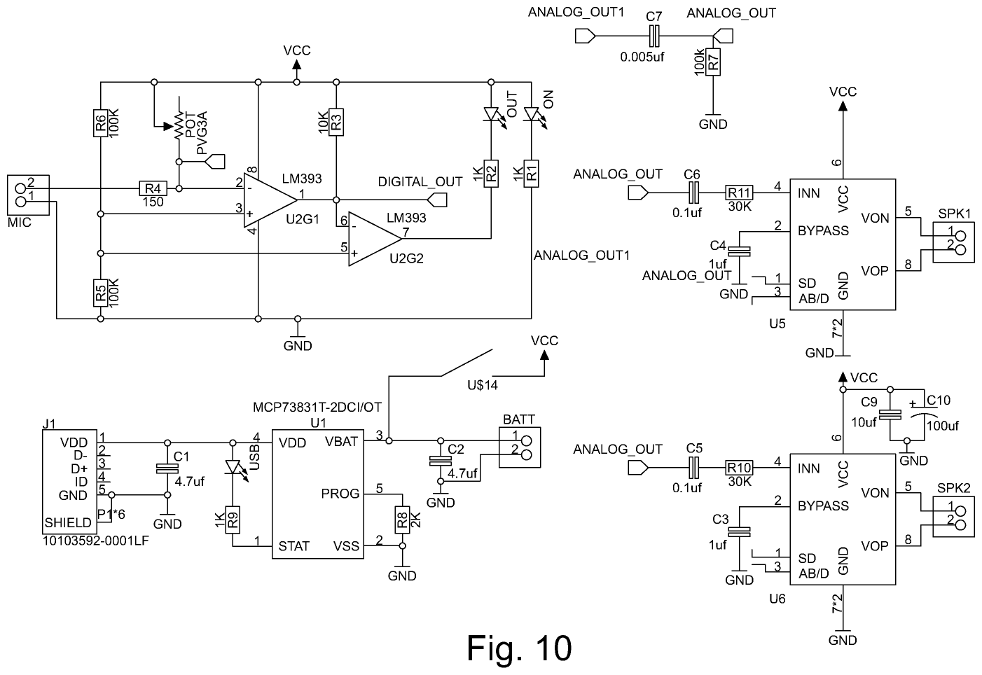

Reference is now made to FIG. 5 and subsequent figures of the drawings, illustrating a further embodiment of the ventilation system for use in a face mask or face goggles which may be utilized in accordance with the present invention. These figures illustrate a mask 200 having a front window 202 including a nose recess 204 and sides 206 each having a connector piece 208 by means of which a strap 210 may be attached to the mask 200. The mask 200 has an inwardly projecting platform 216 and a seal 218, the seal 218 engaging the face of the wearer so that the mask 200 can preferably attached against the face of the wearer in a manner which seals the space defined by the inside of the mask 200 from the outside. Therefore, in a preferred version, air breathed by the wearer will not emanate from any openings or cracks which may be present 222 between the seal 218 and the face of the wearer.

The strap 210 is preferably of the adjustable type, is elasticized, and designed for convenience and comfort. However, there may be more than one strap 210, and the strap may take many different forms or styles.

The platform 216 includes a top ledge 222. A ventilation module 224 is located on the top ledge 222, preferably in a manner which selectively allows it to be secured to or released from the top ledge 222 for maintenance, cleaning, substituting or the like. In one form of the invention, the ventilation module 224 connects to the top ledge 222 in a snap-on fashion, sealing the ventilation module 224 from the top ledge 222 so that air may only be introduced to the inside of the mask or mask interior 226 through designated and proper pathways of the ventilation module 224.

The ventilation module 224 comprises a housing 230. Within the housing 230 there is located a filter 232 behind aperture 234. A motor within the housing 230 produces an air pathway from the outside of the goggles 200 through the filter 232, through an outlet and into the mask interior 226. The housing 230 further comprises a power source such as a battery. The ventilation module 224 is activated by means of a switch 240. A USB port 242 is provided whereby the battery within the ventilation module 224 can be recharged. Further, the ventilation module includes an indicator light 244 indicating that the module is on. As will be seen, the housing 230 of the ventilation module 224 comprises two halves or components which can be separated from each other so that the filter 232 can be cleaned, replaced or otherwise maintained to ensure the optimal efficiency and the effectiveness of the ventilation module 224.

It is within the scope of the invention that air flow between the inside space of the mask and the outside can be in both directions, according to the settings of the motor. As such, air may be introduced into the mask, or selectively removed therefrom, according to the situation. In either situation, fogging and staleness may be reduced or eliminated.

The goggles 200 of the invention may help to prevent liquid splash, saliva, water droplets, bacteria, flying debris, sand or dust from reaching the wearer eyes and face, and can be used in multiple settings including laboratories, mines, construction sites, health environments, to name a few. The mask 200 prevents or reduces fog from condensing on the interior surface thereof, and the cool air may further reduce or remove water vapor and residual heat from the mask interior 226. The lens itself, or the front window 202, may be comprised of a material, or have a material thereon, which itself reduces fogging, and may be comprised of a scratch resistant material as well. Filters such as UV filters may be utilized. The device allows clean air to enter to provide safer air protection, utilizing a positive pressure airflow. Further, the wide sweep of the lens or front window in a preferred embodiment provides an excellent all round peripheral view.

In one embodiment, the battery may have up to 6 to 8 hours of sufficient power to run the motor before it is necessary to recharge. Further, the mask 200 in this embodiment may be sufficiently large to enable the user to wear prescription glasses or other optical devices underneath it. In one embodiment, the motor may have an input voltage of DC5V, and an input power of 0.8 W.

The face mask audio enhancer of the invention may consist of a plastic mask frame, audio PCB and battery. The product may be used with PPE face masks to enhance the audio, so that for example a person that is 6 feet away can be easily heard when speaking through a mask.

In one embodiment, the mask audio enhancer uses a mic, preamp, and two audio power amplifiers. The entire system works from 3.3 volts powered by a Li-ION battery. The battery can be charged using a USB micro cable, similar to those used on any Android or similar phone. The battery can last up to 4 to 8 hours depending on the usage. When speaking into the mic, there may be more power needed from the battery source, and this can reach up to 50 to 100 milliamps. The system is idle around 20 milliamps. The electronic circuit has a potentiometer (POT) to control the amount of gain from the mic. Turning the POT clockwise will increase the gain, while turning counterclockwise will lower the gain. The speakers and mic are arranged and configured to preferably minimize feedback, but feedback may occur if the gain is set too high. The circuit has a high pass filter to amplify mid frequencies which will be heard more easily than lower frequencies. The device objective is to amplify frequencies that are being muffled when wearing a mask, and making speech easier to understand.

To further enhance the amplification of the higher frequencies, two 2-watt speakers may be used with a speaker cage, and with an airtight backing cover to push more air and give the speakers extra volume range of sound. The combination of the mic, preamp, power amp and speaker system, creates the enhanced effect that a voice can easily project through a mask covering.

In one embodiment, the mask frame may be powered on with a small screwdriver, or toothpick. Once powered on, a green LED light will illuminate. The device may also incorporate a peak detection LED that will illuminate if the volume is to loud for the MIC. A regular blade screwdriver that is used with eyeglasses can, for example, be used to adjust the gain level. Optimally, the gain may be adjusted low enough to prevent feedback, but still allowing the audio to pass through.

The product is placed on the face and held on with two straps behind the ears. The PPE face mask is then placed over it, if the product does not already contain a mask barrier. The device may be tested by, for example, standing 6 feet from another person, and talking normally. The mask is designed just to enhance, and not necessarily to cause an increase in volume like a stereo or PA system, although this may in fact be an incorporated feature if appropriate and needed. However, enhancing the lost frequencies is the main objective, so that words are clearly transmitted and can be easily heard and recognized.

The electronic system has the ability to act as a megaphone, if using an external mic that is not close to the speakers. This may be useful for other applications.

The speaker may be powered in many different ways, similar to those discussed above with respect to the microphone assembly.

Variations of the invention are possible, and there are many areas where a different configuration or construction can be used without compromising the effectiveness of the product. One important feature of the ventilated mask and audio enhancement assembly 10 of the invention is its compactness, lightness, as well as the ease by which it can be attached to the user at a convenient location. It can also be attached to a mask directly, so that it has a modular association therewith. It is also simple to use, requiring only simple steps for both assembly and operation.

In the embodiment shown in FIG. 1, the pump 50 is adjacent to or outside of the air conduit 48. However, in other embodiments, the pump may be located within the conduit, and the housing 12 itself may be little more than the conduit with the intake and discharge ports at each end, and preferably a filter 36 for filtering the air to cleanse it of impurities before being conveyed to the ventilation space 22. The pump 50 can, furthermore, be positioned at any convenient location and is not limited to that shown in the figure.

In one embodiment, the housing may be comprised of a light but sturdy plastic material, and the pump contained within these small dimensions. As previously mentioned, a powerful airflow is most often not needed in most applications, and a small pump to convey just small quantities of air into the ventilation space 22 may be sufficient, in most situations, to achieve the objective, namely, the ability to prevent or minimize condensation, to provide a stream of purified fresh air, and to potentially cool the ventilation space 22, all of which contribute to the comfort and convenience of the user. The ventilation mask assembly 10 of the invention is not intended as an oxygen supply for a patient in distress, and the air flow rate is therefore not subject to any rigorous specifications or tolerances. There are many jobs, including those in healthcare, frontline essential businesses, as well as police and firefighting tasks, where the user may have to wear a face mask continually for a long period of time, creating more and more discomfort as time goes by. The ventilator mask assembly 10 of the invention is intended to lessen or diminish these consequences of wearing a face mask, which may, as a result, enable the user to stay on the job longer before requiring a break to recover from the restricted breathing capabilities which follow from the extended wearing of the protective face mask.

Throughout this description, the embodiments and examples shown should be considered as exemplars, rather than limitations on the apparatus and procedures disclosed or claimed. Although many of the examples presented herein involve specific combinations of method acts or system elements, it should be understood that those acts and those elements may be combined in other ways to accomplish the same objectives. Acts, elements and features discussed only in connection with one embodiment are not intended to be excluded from a similar role in other embodiments.

As used herein, “plurality” means two or more. As used herein, a “set” of items may include one or more of such items.

As used herein, whether in the written description or the claims, the terms “comprising”, “including”, “carrying”, “having”, “containing”, “involving”, and the like are to be understood to be open-ended, i.e., to mean including but not limited to. Only the transitional phrases “consisting of” and “consisting essentially of”, respectively, are closed or semi-closed transitional phrases with respect to claims. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. As used herein, “and/or” means that the listed items are alternatives, but the alternatives also include any combination of the listed items.

1. A face mask assembly comprising:
a face mask for placing on the face of a user so as to define a space between the face mask and the face of the user; and
a ventilation component associated with the face mask, the ventilation component comprising a intake port, a discharge port, a pump for conveying air through a conduit from the intake port to the discharge port, an air filter in the conduit, and a power source for the pump;
wherein the discharge port of the ventilation component connects to the face mask so as to deliver air to the space.

2. A face mask assembly as claimed in claim 1 wherein the ventilation component comprises a module which attaches directly to the face mask.

3. A face mask assembly as claimed in claim 2 wherein the face mask comprises a lens and a platform around at least a part of the lens, and wherein the module is mounted upon the platform.

4. A face mask assembly as claimed in claim 2 wherein the power source of the ventilation component is rechargeable, and a USB port connector is provided for connecting the power source to a charger.

5. A face mask assembly as claimed in claim 2 wherein the module is mounted so as to be releasably connectable to the face mask.

6. A face mask assembly as claimed in claim 1, wherein the filter can be selectively removed from the ventilation component for replacement or maintenance.

7. A face mask assembly as claimed in claim 1 further comprising a sound enhancement assembly.

8. A face mask assembly as claimed in claim 7 wherein the face mask has an inner surface and an outer surface, a microphone mounted in or on the inner surface, and a speaker mounted on the outer surface, wherein sounds detected by the microphone are transmitted to the speaker.

9. A face mask assembly as claimed in claim 7 wherein the face mask has an inner surface and an outer surface, a microphone mounted in or on the inner surface, and a transmitter for transmitting sounds detected by the microphone to a remote speaker.

10. A face mask assembly as claimed in claim 8 comprising a power source for the microphone and the speaker.

11. A face mask assembly as claimed in claim 7 wherein the sound enhancement assembly further comprises electronic circuitry for amplifying and enhancing sounds.

12. A face mask assembly comprising:
a face mask for placing on the face of a user so as to define a space between the face mask and the face of the user; and
a sound enhancement assembly associated with the face mask, the sound enhancement assembly comprising a microphone for receiving sounds and speech of the user, and a transmitter for transmitting the sounds and speech of the user to a speaker.

13. A face mask assembly as claimed in claim 12 wherein the microphone is located in a pocket on an inner surface of the face mask.

14. A face mask assembly as claimed in claim 12 further comprising a speaker for receiving sounds from the microphone, wherein the microphone is located on an inner surface of the face mask, and the speaker is located on an outer surface of the face mask.

15. A face mask assembly as claimed in claim 12 further comprising a speaker for receiving sounds from the microphone, wherein the microphone is located on an inner surface of the face mask, and the speaker is located remotely from the microphone.

16. A face mask assembly as claimed in claim 15 wherein the microphone and speaker are connected wirelessly or by Bluetooth.

17. A face mask assembly as claimed in claim 12 wherein the face mask comprises a frame member, the frame member holding the microphone and speakers, the frame member further holding a power source and electronic circuitry.

18. A face mask assembly as claimed in claim 17 wherein the frame member comprises a base which sits against the face of the wearer, and a plurality of posts forming the shape of a mask, and further comprising a mask member, which may be releasably placed on the frame member to define an inner closed space.

19. A method of masking a user comprising:
placing a mask over the face of the user;
connecting the mask to a ventilation component;
activating the ventilation component to provide a stream of air between the outside of the mask and the inside thereof;
filtering the stream of air so that the air exits the ventilation component in a filtered condition; and
placing a sound enhancement assembly on the mask so that the sounds and speech of the user acquire improved quality to a listener.

20. A method as claimed in claim 19 wherein the placing of the sound enhancement assembly comprises placing a microphone on and inside portion of the mask, and locating a speaker outside of the mask either directly adjacent thereto or remotely therefrom.
